(19) United States
(12) Patent Application Publication
(10) Pub. No.: US 2008/0220979 A1
(43) Pub. Date: Sep. 11, 2008

(54) Rapid Method To Detect Nucleic Acid Molecules

(75) Inventors: Dong Wang, Beijing (CN); Gang Li, Beijing (CN); Xuemei Ma, Beijing (CN); Chengxun Liu, Beijing (CN); Yuxiang Zhou, Beijing (CN); Jing Cheng, Beijing (CN)

Correspondence Address: MORRISON & FOERSTER LLP, 12531 HIGH BLUFF DRIVE, SUITE 100, SAN DIEGO, CA, 92130-2040, US

(73) Assignees: CAPITALBIO CORPORATION, Beijing (CN); TSINGHUA UNIVERSITY, Beijing (CN)

(21) Appl. No.: 10/568,206

(22) PCT Filed: Aug. 27, 2003
(86) PCT No.: PCT/CN2003/000722

§ 371 Date: Aug. 6, 2007

(30) Foreign Application Priority Data
Aug. 13, 2003 (CN) 03153279.9

(51) Int. Cl. C40B 30/04; C12Q 1/68

(52) U.S. Cl. 506/9; 435/6

(57) ABSTRACT

This invention relates to the field of detecting nucleic acid molecules using microarrays. The invention provides a method for detecting a target nucleic acid molecule in a biological sample by hybridizing a cell lysate directly probes immobilized on microarrays without any nucleic acid purification.

### BACKGROUND OF THE INVENTION

1. Field of the Invention

The present invention relates to a rapid method to detect nucleic acids molecules on microarrays.

2. Description of the Related Art

Hybridization between nucleic acids molecules is a useful tool to detect target nucleic acid sequences in biological research and clinical medicine. To prepare for hybridization, it is usually necessary to isolate or purify nucleic acids from other cellular components. The isolation or purification process requires a variety of equipments (e.g., centrifuge, refrigerator, and electrophoresis equipment) and is time-consuming. The process often takes hours or even days, and is not useful for rapid nucleic acid detection. Although several automatic workstations for extracting and purifying nucleic acids from cell lysate, such as Biorobot 9600 and Biorobot 9604 (Qiagen), have been developed, these machines are expensive and still need a relative long time for the purification of one sample.

Since Dr. Fodor first reported DNA chip on the journal of Science in 1991, the DNA chip or biochip technology has developed rapidly (Fodor et al., Science 251:767-773 (1991); Marshall et al. Nat. Biotechnol. 16:27-31 (1998)). A variety of biochips have been developed (Cheng et al., Mol. Diagn. 1 :183-200 (1996)) and they are playing an important role in life science researches.

Biochemical reactions and analyses often include three steps: sample preparation, biochemical reactions and signal detection and data analyses. Efforts have been made to perform all steps of biochemical analysis on chips to produce micro-analysis systems or lab-on-chip systems. Using such micro-analysis systems or lab-on-chip systems, it will be possible to complete all analytic steps from sample preparation to obtain analytical results in a closed system rapidly.

One of the difficulties in achieving “lab-on-chip” systems is the nucleic acids extraction and purification, which not only takes a long time but also is difficult to be managed in a micro-device. Therefore, there is a need to overcome this limitation.

In 1998, Cheng et al developed a method for preparing nucleic acid from E. Coli and performed hybridization analysis on DNA chip (Cheng et al. Nature Biotechnology 16:541-546 (1998)). The method allows hybridization of the bacterial lysate with DNA chip by lysing bacteria with an electronic pulse, then diluting and digesting the bacterial lysate with proteinase K. This is the first time that an integration of sample preparation, biological reaction, and detection have been achieved. However, the method still requires the step of removing proteins from the cell lysate with proteinase.

The rapid detection of nucleic acids molecule is important for research in life science and clinical diagnosis, especially in clinical diagnosis of infectious diseases. For example, the detection of infectious bacteria in hospital needs culture, pure culture and several biochemical detections, which takes several days and is disadvantageous for patient. The present invention provides a rapid method which takes no more than 90 minutes to get a clear and accurate result. The present invention only takes two steps (the lysis of biological samples and hybridization with microarrays to get clearly results, and can be easily applied in miniaturization and automation systems.

### SUMMARY OF THE INVENTION

The object of the present invention is to provide a rapid method to detect nucleic acids by the direct hybridization of cellular lysate with microarrays without any further purification. This method is simple, low-cost, convenient-to-operate, contamination-free, and easy-to-integrate.

In an exemplary embodiment, the cellular lysate can be hybridized directly with probes on microarrays without any further purification; and therefore, the whole procedure of this method is simple and time-saving. In this method, the cell sample is firstly lysed by physical, chemical or biological method in a lysis buffer, which contains material to label the target nucleic acids; then, the cellular lysate is hybridized with microarrays without any further purification to detect the target nucleic acids sequences.

In one aspect, the present invention is directed to a method for detecting a target nucleic acid molecule, said method comprises: a) preparing a cell lysate comprising lysing a cell in a biological sample in a lysis buffer to release the target nucleic acid molecule from the cell; b) incubating the cell lysate from step a), without nucleic acid purification, with a nucleic acid probe immobilized on a solid substrate under conditions that allow hybridization between the target nucleic acid molecule and the probe, wherein the nucleic acid probe comprises a sequence complementary to the target nucleic acid molecule; c) assessing hybridization between the target nucleic acid molecule and the probe to determine the presence, absence and/or amount of the target nucleic acid molecule.

### BRIEF DESCRIPTION OF THE DRAWING

FIG. 1 illustrates the result of hybridization in the example.

### DETAILED DESCRIPTION OF THE INVENTION

For clarity of disclosure, and not by way of limitation, the detailed description of the invention is divided into the subsections that follow.

### A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “primer” refers to an oligonucleotide that hybridizes to a target sequence, typically to prime the nucleic acid in the amplification process.

As used herein, “probe” refers to an oligonucleotide that hybridizes to a target sequence, typically to facilitate its detection. The term “target sequence” or “target nucleic acid molecule” refers to a nucleic acid sequence to which the probe specifically binds. Unlike a primer that is used to prime the target nucleic acid in the amplification process, a probe need not be extended to amplify target sequence using a polymerase enzyme. However, it will be apparent to those skilled in the art that probes and primers are structurally similar or identical in many cases.

As used herein, “sample” refers to anything which may contain an analyte to be analyzed using the present devices and/or methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, “without nucleic acid purification” means that after a cell in a biological sample is lysed in a lysis buffer, nucleic acid molecules released from the cell are not purified, isolated, or extracted from the lysate before they are hybridized to probes immobilized on a solid support.

As used herein, “complementary or matched” means that two nucleic acid sequences have at least 50% sequence identity. Preferably, the two nucleic acid sequences have at least 60%, 70,%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein: “stringency of hybridization” in determining percentage mismatch is as follows:

1) high stringency: 0.1×SSPE (or 0.1×SSC), 0.1% SDS, 65° C.;

2) medium stringency: 0.2×SSPE (or 1.0×SSC), 0.1% SDS, 50° C. (also referred to as moderate stringency); and

3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “melting temperature” (“Tm”) refers to the midpoint of the temperature range over which nucleic acid duplex, i.e., DNA:DNA, DNA:RNA, RNA:RNA, PNA:DNA, LNA:RNA and LNA:DNA, etc., is denatured.

As used herein the term “assessing” is intended to include quantitative and/or qualitative determination of an analyte present in the sample, and also of obtaining an index, ratio, percentage, visual or other value indicative of the level of the analyte in the sample. Assessment may be direct or indirect and the chemical species actually detected need not of course be the analyte itself but may for example be a derivative thereof or some further substance.

### B. Methods for Detecting Target Nucleic Acid Molecules

In one aspect, the present invention provides a method for detecting a target nucleic acid molecule, said method comprises: a) preparing a cell lysate comprising lysing a cell in a biological sample in a lysis buffer to release the target nucleic acid molecule from the cell; b) incubating the cell lysate from step a), without nucleic acid purification, with a nucleic acid probe immobilized on a solid substrate under conditions that allow hybridization between the target nucleic acid molecule and the probe, wherein the nucleic acid probe comprises a sequence complementary to the target nucleic acid molecule; c) assessing hybridization between the target nucleic acid molecule and the probe to determine the presence, absence and/or amount of the target nucleic acid molecule.

The method of the invention can be generally used in nucleic acid detections, for example, detection and identification of clinical bacteria, detection of drug-resistant bacteria, environmental detection, forensic detection, and analysis of gene expression, etc.

Preparation of Cell Lysates

Target nucleic acid molecules in any biological samples can be detected using the method described herein. Any suitable biological samples, including samples of human, animal, or environmental (e.g., soil or water) origin, can be analyzed using the present method. Biological samples can include body fluids, such as urine, blood, semen, cerebrospinal fluid, pus, amniotic fluid, tears, or semisolid or fluid discharge, e.g., sputum, saliva, lung aspirate, vaginal or urethral discharge, stool or solid tissue samples, such as a biopsy or chorionic villi specimens. Biological samples also include samples collected with swabs from the skin, genitalia, or throat. In some embodiments, the biological sample is a non-virus biological organism, a biological tissue, a eukaryotic cell, or a prokaryotic cell.

A cell in a biological sample containing the target nucleic acid molecule can be lysed in a lysis buffer using any known methods, such as a physical method, a chemical method, a biological method, or any combination thereof. Exemplary physical methods include grinding, ultrasonic lysing, lysing with high temperature, and freezing. Exemplary chemical methods include lysing with a protein denaturant or a detergent. Exemplary biological methods include lysing with a proteinase or a lysozyme.

In some embodiments, the cell lysate prepared comprises an agent selected from the group consisting of a detergent, a protein denaturant, a buffer, a nuclease inhibitor, a salt, and a combination thereof.

The target nucleic acid molecule of the invention can be a genomic DNA, a plasmid, a mitochondria DNA, a chloroplast DNA, a messenger RNA, a ribosomal RNA, and a small nuclear RNA.

Hybridization Conditions

The cell lysate prepared as describe above can be incubated, without nucleic acid purification or extraction, with a nucleic acid probe immobilized on a solid substrate under conditions that allow hybridization between the target nucleic acid molecule and the probe.

In some embodiments, the cell lysate is in incubated with the probe immobilized on the substrate in the lysis buffer for hybridization.

In other embodiments, an agent that aids for hybridization is added to the cell lysate before the cell lysate is incubated with the probe. Such agent can be NaCl, citrate sodium, and SDS.

Hybridization can be carried out under any suitable technique known in the art. It will be apparent to those skilled in the art that hybridization conditions can be altered to increase or decrease the degree of hybridization, the level of specificity of the hybridization, and the background level of non-specific binding (i.e., by altering hybridization or wash salt concentrations or temperatures). The hybridization between the probe and the target nucleotide sequence can be carried out under any suitable stringencies, including high, middle or low stringency. Typically, hybridizations will be performed under conditions of high stringency.

Hybridization between the probe and target nucleic acids can be homogenous, e.g., typical conditions used in molecular beacons (Tyagi S. et al., Nature Biotechnology, 14:303-308 (1996); and U.S. Pat. No. 6,150,097) and in hybridization protection assay (Gen-Probe, Inc) (U.S. Pat. No. 6,004,745), or heterogeneous (typical conditions used in different type of nitrocellulose based hybridization and those used in magnetic bead based hybridization).

The target polynucleotide sequence may be detected by hybridization with an oligonucleotide probe that forms a stable hybrid with that of the target sequence under high to low stringency hybridization and wash conditions. An advantage of detection by hybridization is that, depending on the probes used, additional specificity is possible. If it is expected that the probes will be completely complementary (i.e., about 99% or greater) to the target sequence, high stringency conditions will be used. If some mismatching is expected, for example, if variant strains are expected with the result that the probe will not be completely complementary, the stringency of hybridization may be lessened. However, conditions are selected to minimize or eliminate nonspecific hybridization.

Conditions those affect hybridization and those select against nonspecific hybridization are known in the art (Molecular Cloning A Laboratory Manual, second edition, J. Sambrook, E. Fritsch, T. Maniatis, Cold Spring Harbor Laboratory Press, 1989). Generally, lower salt concentration and higher temperature increase the stringency of hybridization. For example, in general, stringent hybridization conditions include incubation in solutions that contain approximately 0.1×SSC, 0.1% SDS, at about 65° C. incubation/wash temperature. Middle stringent conditions are incubation in solutions that contain approximately 1-2×SSC, 0.1% SDS and about 50° C.-65° C. incubation/wash temperature. The low stringency conditions are 2×SSC and about 30° C.-50° C.

An alternate method of hybridization and washing is first to carry out a low stringency hybridization (5×SSPE, 0.5% SDS) followed by a high stringency wash in the presence of 3M tetramethyl-ammonium chloride (TMAC). The effect of the TMAC is to equalize the relative binding of A-T and G-C base pairs so that the efficiency of hybridization at a given temperature corresponds more closely to the length of the polynucleotide. Using TMAC, it is possible to vary the temperature of the wash to achieve the level of stringency desired (Wood et al., Proc. Natl. Acad. Sci. USA, 82:1585-1588 (1985)).

A hybridization solution may contain 25% formamide, 5×SSC, 5× Denhardt's solution, 100 μg/ml of single stranded DNA, 5% dextran sulfate, or other reagents known to be useful for probe hybridization.

Probes

The invention provides a nucleic acid probe immobilized on a solid substrate which comprises a sequence complementary to the target nucleic acid molecule.

In some embodiments, the nucleic acid probe immobilized on the solid substrate comprises a single-stranded oligonucleotide or double-stranded PCR product.

The oligonucleotide probes can be produced by any suitable method. For example, the probes can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can also be prepared by using the triester method of Matteucci et al., J. Am. Chem. Soc., 3:3185-3191 (1981). Alternatively, automated synthesis may be preferred, for example, on a Applied Biosynthesis DNA synthesizer using cyanoethyl phosphoramidite chemistry. Preferably, the probes are chemically synthesized.

Suitable bases for preparing the oligonucleotide probes of the present invention may be selected from naturally occurring nucleotide bases such as adenine, cytosine, guanine, uracil, and thymine. It may also be selected from nonnaturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylpseudouridine, beta-D-galactosylqueosine, 2′-Omethylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N6-isopentenyladenosine, N-((9-.beta.-D-ribofuranosyl-2-methylthiopurine-6-yl)carbamoyl)threonine, N-((9-beta-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-end cap” strategy by which nuclease-resistant linkages are substituted for phosphodiester linkages at the 3′ end of the oligonucleotide (Shaw et al., Nucleic Acids Res., 19:747 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem., 36:1923 (1993)). Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (MOMI) linkages. Phosphorothioate and methylphosphonate-modified oligonucleotides are particularly preferred due to their availability through automated oligonucleotide synthesis. The oligonucleotide may be a “peptide nucleic acid” such as described by (Milligan et al., J. Med. Chem., 36:1923 (1993)). The only requirement is that the oligonucleotide probe should possess a sequence at least a portion of which is capable of binding to a portion of the sequence of a target DNA molecule.

Hybridization probes can be of any suitable length. There is no lower or upper limits to the length of the probe, as long as the probe hybridizes to the target nucleic acids and functions effectively as a probe (e.g., facilitates detection). The probes of the present invention can be as short as 50, 40, 30, 20, 15, or 10 nucleotides, or shorter. Likewise, the probes can be as long as 20, 40, 50, 60, 75, 100 or 200 nucleotides, or longer, e.g., to the full length of the target sequence. Generally, the probes will have at least 14 nucleotides, preferably at least 18 nucleotides, and more preferably at least 20 to 30 nucleotides of either of the complementary target nucleic acid strands and does not contain any hairpin secondary structures. In specific embodiments, the probe can have a length of at least 30 nucleotides or at least 50 nucleotides. If there is to be complete complementarity, i.e., if the strand contains a sequence identical to that of the probe, the duplex will be relatively stable under even stringent conditions and the probes may be short, i.e., in the range of about 10-30 base pairs. If some degree of mismatch is expected in the probe, i.e., if it is suspected that the probe would hybridize to a variant region, or to a group of sequences such as all species within a specific genus, the probe may be of greater length (i.e., 15-40 bases) to balance the effect of the mismatch(es).

Immobilization of Probes

The probes can be immobilized on a solid substrate or support, such as a nylon film, a pyroxylin film, a silicon, a glass, a ceramic, a metal, a plastic, and a combination thereof. Other suitable solid substrate or support includes rubber or polymer surface. The probe may also be immobilized in a 3-dimensional porous gel substrate, e.g., Packard HydroGel chip (Broude et al., Nucleic Acids Res., 29(19):E92 (2001)).

In some embodiments, the solid substrate comprises a plurality of nucleic acid probes, and the plurality of the nucleic acid probes are immobilized on the solid substrate to form an array.

In some embodiments, the plurality of the nucleic acid probes have different nucleotide sequences. In some embodiments, the number of different probes is from about 2 to about 100,000.

In some embodiments, the area of the array is from about 0.01 mm² to about 100 cm².

In some embodiments, the array is a two-dimensional array, a three-dimensional array, or a four-dimensional array.

For an array-based assay, the probes are preferably immobilized to a solid support such as a “biochip”. The solid support may be biological, nonbiological, organic, inorganic, or a combination of any of these, existing as particles, strands, precipitates, gels, sheets, tubing, spheres, containers, capillaries, pads, slices, films, plates, slides, etc.

A microarray biochip containing a library of probes can be prepared by a number of well known approaches including, for example, light-directed methods, such as VLSIPS™ described in U.S. Pat. Nos. 5,143,854, 5,384,261 or 5,561,071; bead based methods such as described in U.S. Pat. No. 5,541,061; and pin based methods such as detailed in U.S. Pat. No. 5,288,514. U.S. Pat. No. 5,556,752, which details the preparation of a library of different double stranded probes as a microarray using the VLSIPS™, is also suitable for preparing a library of hairpin probes in a microarray.

Flow channel methods, such as described in U.S. Pat. Nos. 5,677,195 and 5,384,261, can be used to prepare a microarray biochip having a variety of different probes. In this case, certain activated regions of the substrate are mechanically separated from other regions when the probes are delivered through a flow channel to the support. A detailed description of the flow channel method can be found in U.S. Pat. No. 5,556,752, including the use of protective coating wetting facilitators to enhance the directed channeling of liquids though designated flow paths.

Spotting methods also can be used to prepare a microarray biochip with a variety of probes immobilized thereon. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much probe or other reagent as necessary at each stop. Typical dispensers include micropipettes, nanopippettes, ink-jet type cartridges and pins to deliver the probe containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously. Spotting methods are well known in the art and include, for example, those described in U.S. Pat. Nos. 5,288,514, 5,312,233 and 6,024,138. In some cases, a combination of flow channels and “spotting” on predefined regions of the support also can be used to prepare microarray biochips with immobilized probes.

A solid support for immobilizing probes is preferably flat, but may take on alternative surface configurations. For example, the solid support may contain raised or depressed regions on which probe synthesis takes place or where probes are attached. In some embodiments, the solid support can be chosen to provide appropriate light-absorbing characteristics. For example, the support may be a polymerized Langmuir Blodgett film, glass or functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art.

The surface of the solid support can contain reactive groups, which include carboxyl, amino, hydroxyl, thiol, or the like, suitable for conjugating to a reactive group associated with an oligonucleotide or a nucleic acid. Preferably, the surface is optically transparent and will have surface Si—OH functionalities, such as those found on silica surfaces.

The probes can be attached to the support by chemical or physical means such as through ionic, covalent or other forces well known in the art. Immobilization of nucleic acids and oligonucleotides can be achieved by any means well known in the art (see, e.g., Dattagupta et al., Analytical Biochemistry, 177:85-89 (1989); Saiki et al., Proc. Natl. Acad. Sci. USA, 86:6230-6234 (1989); and Gravitt et al., J. Clin. Micro., 36:3020-3027 (1998)).

The probes can be attached to a support by means of a spacer molecule, e.g., as described in U.S. Pat. No. 5,556,752 to Lockhart et al., to provide space between the double stranded portion of the probe as may be helpful in hybridization assays. A spacer molecule typically comprises between 6-50 atoms in length and includes a surface attaching portion that attaches to the support. Attachment to the support can be accomplished by carbon-carbon bonds using, for example, supports having (poly)trifluorochloroethylene surfaces, or preferably, by siloxane bonds (using, for example, glass or silicon oxide as the solid support). Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful are surface attaching groups.

The spacer can also include an extended portion or longer chain portion that is attached to the surface-attaching portion of the probe. For example, amines, hydroxyl, thiol, and carboxyl groups are suitable for attaching the extended portion of the spacer to the surface-attaching portion. The extended portion of the spacer can be any of a variety of molecules which are inert to any subsequent conditions for polymer synthesis. These longer chain portions will typically be aryl acetylene, ethylene glycol oligomers containing 2-14 monomer units, diamines, diacids, amino acids, peptides, or combinations thereof.

In some embodiments, the extended portion of the spacer is a polynucleotide or the entire spacer can be a polynucleotide. The extended portion of the spacer also can be constructed of polyethyleneglycols, polynucleotides, alkylene, polyalcohol, polyester, polyamine, polyphosphodiester and combinations thereof. Additionally, for use in synthesis of probes, the spacer can have a protecting group attached to a functional group (e.g., hydroxyl, amino or carboxylic acid) on the distal or terminal end of the spacer (opposite the solid support). After deprotection and coupling, the distal end can be covalently bound to an oligomer or probe.

The present method can be used to analyze a single sample with a single probe at a time. Preferably, the method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously.

Detection of the Hybrid

Detection of hybridization between the probe and the target nucleic acids can be carried out by any method known in the art, e.g., labeling the probe, the secondary probe (or reporter), the target nucleic acids or some combination thereof, and are suitable for purposes of the present invention. Alternatively, the hybrid may be detected by mass spectroscopy in the absence of detectable label (e.g., U.S. Pat. No. 6,300,076).

The detectable label is a moiety that can be detected either directly or indirectly after the hybridization. In other words, a detectable label has a measurable physical property (e.g., fluorescence or absorbance) or is participant in an enzyme reaction. Using direct labeling, the target nucleotide sequence or the probe is labeled, and the formation of the hybrid is assessed by detecting the label in the hybrid. Using indirect labeling, a secondary probe is labeled, and the formation of the hybrid is assessed by the detection of a secondary hybrid formed between the secondary probe and the original hybrid.

Methods of labeling probes or nucleic acids are well known in the art. Suitable labels include fluorophores, chromophores, luminophores, radioactive isotopes, electron dense reagents, FRET (fluorescence resonance energy transfer), enzymes and ligands having specific binding partners. Particularly useful labels are enzymatically active groups such as enzymes (Wisdom, Clin Chem., 22:1243 (1976)); enzyme substrates (British Pat. No. 1,548,741); coenzymes (U.S. Pat. Nos. 4,230,797 and 4,238,565) and enzyme inhibitors (U.S. Pat. No. 4,134,792); fluorescers (Soini and Hemmila, Clin. Chem., 25:353 (1979)); chromophores including phycobiliproteins, luminescers such as chemiluminescers and bioluminescers (Gorus and Schram, Clin. Chem., 25:512 (1979) and ibid, 1531); specifically bindable ligands, i.e., protein binding ligands; antigens; and residues comprising radioisotopes such as ³H, ³⁵S, ³²P, ¹²⁵I, and ¹⁴C. Such labels are detected on the basis of their own physical properties (e.g., fluorescers, chromophores and radioisotopes) or their reactive or binding properties (e.g., antibodies, enzymes, substrates, coenzymes and inhibitors). Ligand labels are also useful for solid phase capture of the oligonucleotide probe (i.e., capture probes). Exemplary labels include biotin (detectable by binding to labeled avidin or streptavidin) and enzymes, such as horseradish peroxidase or alkaline phosphatase (detectable by addition of enzyme substrates to produce a colored reaction product).

For example, a radioisotope-labeled probe or target nucleic acid can be detected by autoradiography. Alternatively, the probe or the target nucleic acid labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As a further alternative, the probe or nucleic acid may be labeled with a moiety that requires additional reagents to detect the hybridization. If the label is an enzyme, the labeled nucleic acid, e.g., DNA, is ultimately placed in a suitable medium to determine the extent of catalysis. For example, a cofactor-labeled nucleic acid can be detected by adding the enzyme for which the label is a cofactor and a substrate for the enzyme. Thus, if the enzyme is a phosphatase, the medium can contain nitrophenyl phosphate and one can monitor the amount of nitrophenol generated by observing the color. If the enzyme is a beta-galactosidase, the medium can contain o-nitro-phenyl-D-galacto-pyranoside, which also liberates nitrophenol. Exemplary examples of the latter include, but are not limited to, beta-galactosidase, alkaline phosphatase, papain and peroxidase. For in situ hybridization studies, the final product of the substrate is preferably water insoluble. Other labels, e.g., dyes, will be evident to one having ordinary skill in the art.

The label can be linked directly to the DNA binding ligand, e.g., acridine dyes, phenanthridines, phenazines, furocoumarins, phenothiazines and quinolines, by direct chemical linkage such as involving covalent bonds, or by indirect linkage such as by the incorporation of the label in a microcapsule or liposome, which in turn is linked to the binding ligand. Methods by which the label is linked to a DNA binding ligand such as an intercalator compound are well known in the art and any convenient method can be used. Representative intercalating agents include mono-or bis-azido aminoalkyl methidium or ethidium compounds, ethidium monoazide ethidium diazide, ethidium dimer azide (Mitchell et al., J. Am. Chem. Soc., 104:4265 (1982))), 4-azido-7-chloroquinoline, 2-azidofluorene, 4′-aminomethyl-4,5′-dimethylangelicin, 4′-aminomethyl-trioxsalen (4′aminomethyl-4,5′,8-trimethyl-psoralen), 3-carboxy-5- or -8-amino- or -hydroxy-psoralen. A specific nucleic acid binding azido compound has been described by Forster et al., Nucleic Acid Res., 13:745 (1985). Other useful photoreactable intercalators are the furocoumarins which form (2+2) cycloadducts with pyrimidine residues. Alkylating agents also can be used as the DNA binding ligand, including, for example, bis-chloroethylamines and epoxides or aziridines, e.g., aflatoxins, polycyclic hydrocarbon epoxides, mitomycin and norphillin A. Particularly useful photoreactive forms of intercalating agents are the azidointercalators. Their reactive nitrenes are readily generated at long wavelength ultraviolet or visible light and the nitrenes of arylazides prefer insertion reactions over their rearrangement products (White et al., Meth. Enzymol., 46:644 (1977)).

The probe may also be modified for use in a specific format such as the addition of 10-100 T residues for reverse dot blot or the conjugation to bovine serum albumin or immobilization onto magnetic beads.

When detecting hybridization by an indirect detection method, a detectably labeled second probe(s) (or reporter) can be added after initial hybridization between the probe and the target or during hybridization of the probe and the target. Optionally, the hybridization conditions may be modified after addition of the secondary probe (or reporter). After hybridization, unhybridized secondary probe can be separated from the initial probe, for example, by washing if the initial probe is immobilized on a solid support. In the case of a solid support, detection of label bound to locations on the support indicates hybridization of a target nucleotide sequence in the sample to the probe.

The detectably labeled secondary probe (or reporter) can be a specific probe. Alternatively, the detectably labeled probe can be a degenerate probe, e.g., a mixture of sequences such as whole genomic DNA essentially as described in U.S. Pat. No. 5,348,855. In the latter case, labeling can be accomplished with intercalating dyes if the secondary probe contains double stranded DNA. Preferred DNA-binding ligands are intercalator compounds such as those described above.

A secondary probe also can be a library of random nucleotide probe sequences. The length of a secondary probe should be decided in view of the length and composition of the primary probe or the target nucleotide sequence on the solid support that is to be detected by the secondary probe. Such a probe library is preferably provided with a 3′ or 5′ end labeled with photoactivatable reagent and the other end loaded with a detection reagent such as a fluorophore, enzyme, dye, luminophore, or other detectably known moiety.

The particular sequence used in making the labeled nucleic acid can be varied. Thus, for example, an amino-substituted psoralen can first be photochemically coupled with a nucleic acid, the product having pendant amino groups by which it can be coupled to the label, i.e., labeling is carried out by photochemically reacting a DNA binding ligand with the nucleic acid in the test sample. Alternatively, the psoralen can first be coupled to a label such as an enzyme and then to the nucleic acid.

Advantageously, the DNA binding ligand is first combined with label chemically and thereafter combined with the nucleic acid probe. For example, since biotin carries a carboxyl group, it can be combined with a furocoumarin by way of amide or ester formation without interfering with the photochemical reactivity of the furocoumarin or the biological activity of the biotin. Aminomethylangelicin, psoralen and phenanthridium derivatives can similarly be linked to a label, as can phenanthridium halides and derivatives thereof such as aminopropyl methidium chloride (Hertzberg et al, J. Amer. Chem. Soc., 104:313 (1982)). Alternatively, a bifunctional reagent such as dithiobis succinimidyl propionate or 1,4-butanediol diglycidyl ether can be used directly to couple the DNA binding ligand to the label where the reactants have alkyl amino residues, again in a known manner with regard to solvents, proportions and reaction conditions. Certain bifunctional reagents, possibly glutaraldehyde may not be suitable because, while they couple, they may modify nucleic acid and thus interfere with the assay. Routine precautions can be taken to prevent such difficulties.

Also advantageously, the DNA binding ligand can be linked to the label by a spacer, which includes a chain of up to about 40 atoms, preferably about 2 to 20 atoms, including, but not limited to, carbon, oxygen, nitrogen and sulfur. Such spacer can be the polyfunctional radical of a member including, but not limited to, peptide, hydrocarbon, polyalcohol, polyether, polyamine, polyimine and carbohydrate, e.g., -glycyl-glycyl-glycyl- or other oligopeptide, carbonyl dipeptides, and omega-amino-alkane-carbonyl radical or the like. Sugar, polyethylene oxide radicals, glyceryl, pentaeryiritol, and like radicals also can serve as spacers. Spacers can be directly linked to the nucleic acid-binding ligand and/or the label, or the linkages may include a divalent radical of a coupler such as dithiobis succinimidyl propionate, 1,4-butanediol diglycidyl ether, a diisocyanate, carbodiimide, glyoxal, glutaraldehyde, or the like.

Secondary probe for indirect detection of hybridization can be also detected by energy transfer such as in the “beacon probe” method described by Tyagi and Kramer, Nature Biotech, 14:303-309 (1996) or U.S. Pat. Nos. 5,119,801 and 5,312,728 to Lizardi et al. Any FRET detection system known in the art can be used in the present method. For example, the AlphaScreen™ system can be used. AlphaScreen technology is an “Amplified Luminescent Proximity Homogeneous Assay” method. Upon illumination with laser light at 680 nm, a photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity of the donor bead, by virtue of a biological interaction, the singlet-state oxygen molecules reacts with chemiluminescent groups in the acceptor beads, which immediately transfer energy to fluorescent acceptors in the same bead. These fluorescent acceptors shift the emission wavelength to 520-620 nm. The whole reaction has a 0.3 second half-life of decay, so measurement can take place in time-resolved mode. Other exemplary FRET donor/acceptor pairs include Fluorescein (donor) and tetramethylrhodamine (acceptor) with an effective distance of 55 Å; IAEDANS (donor) and Fluorescein (acceptor) with an effective distance of 46 Å; and Fluorescein (donor) and QSY-7 dye (acceptor) with an effective distance of 61 Å (Molecular Probes).

Quantitative assays for nucleic acid detection also can be performed according to the present invention. The amount of secondary probe bound to a microarray spot can be measured and can be related to the amount of nucleic acid target which is in the sample. Dilutions of the sample can be used along with controls containing known amount of the target nucleic acid. The precise conditions for performing these steps will be apparent to one skilled in the art. In microarray analysis, the detectable label can be visualized or assessed by placing the probe array next to x-ray film or phosphoimagers to identify the sites where the probe has bound. Fluorescence can be detected by way of a charge-coupled device (CCD) or laser scanning.

In some embodiments, the hybridization between the target nucleic acid molecule and the nucleic acid probe is assessed by determining binding of a reporter to the target nucleic acid molecule, wherein the reporter comprises a detectable marker selected from the group consisting of a fluorescein, an isotope, a biotin, a digoxin, a gold colloid, a magnetic bead, a electrochemical label, and a chemiluminescent label.

### C. EXAMPLE

The present example illustrates a rapid method to detect nucleotide acids molecules on microarrays. In the present example, the biological samples was first lysed with a lysing buffer through physical, chemical or biological method, then the lysate was hybridized directly to microarrays without any further nucleic acids purification. Compared with conventional methods to detect nucleic acids, this rapid method provides simple, easy-to-operate and time-saving processing. This exemplary method may have many application, e.g., bacteria/cell detection.

### Rapid Bacterial Detection and Identification on Microarrays

Materials

1) Overnight culture of Staphylococcus aureus (concentration 1.6×10⁹ cfu/mL)

2) 20×SSPE: 3.6M NaCl, 0.2M phosphate buffer, pH 7.4, 20 mM EDTA

3) Lysis buffer: 6% SDS, 0.1 Tris, 0.05M EDTA, and 4 ng/μL Hex labeled reporter probes

4) Washing buffer: 2×SSPE, 0.1% SDS

5) Four bacterial species-specific probes were tethered on the surface of a microarray having aldehyde groups as capture probes. The distance between each spot of probes is 300 [symbol] m and the diameter of each spot is 150 [symbol] m. The sequences of capture probes and reporter probes were listed in Table 1.

TABLE 1

Nucleotide sequences of probes and
reporter probes

| | | |
|---|---|---|
| Bacterial | Sequence 5′-3′ of | Sequence 5′-3′ of |
| species | capture probes | reporter probes |

| | | |
|---|---|---|
| E. coli | NH₂-T₁₂-GTATTAACTT | TTCCTCCCCGCTGAAAGT |
| | TACTCCC | ACTTTAC-Hex |
| S. aureus | NH₂-T₁₂-AGCAAGCTTC | TTCGCTCGACTTGCATGT |
| | TCGTCCG | ATTAGGC-Hex |
| P. aeruginosa | NH₂-T₁₂-GCGCCCGTTT | GTTATCCCCCACTACCAG |
| | CCGGAC | GCAGATTCC-Hex |
| S. pyogenes | NH₂-T₁₂-ATTACTAACA | GTCTCTCTTATGCGGTAT |
| | TGCGTTA | TAGCTA-Hex |

Methods

1) One mL of S. aureus overnight cultures with concentration of 1.6×10⁸, 1.6×10⁷, 1.6×10⁶, 1.6×10⁵, 1.6×10⁴, 1.6×10³ cfu/mL were centrifuged for 5 min at 10,000 rpm, and the supernatants were discarded.

2) The cells were resuspended in 20 μL lysis buffer.

3) The cells resuspended were sonicated for 2 min with 300 mV and 990 KHz to lyse bacterial cells. The 16S rRNA released from the bacterial cells were allowed to hybridize with the florescent reporters in the lysis buffer so that the target 16S rRNA was labeled.

4) Then, 2 μL of 20×SSPE were added to the cell lysate and mixed.

5) The cell lysate (10 μL) was added on a microarray and was incubated for 1 hr at 42° C.

6) The microarray was washed for 15 min with the washing buffer and centrifuged to spin off the liquid.

7) The microarray was then scanned on Genepix Scanner and data collected were analyzed.

Results and Discussion

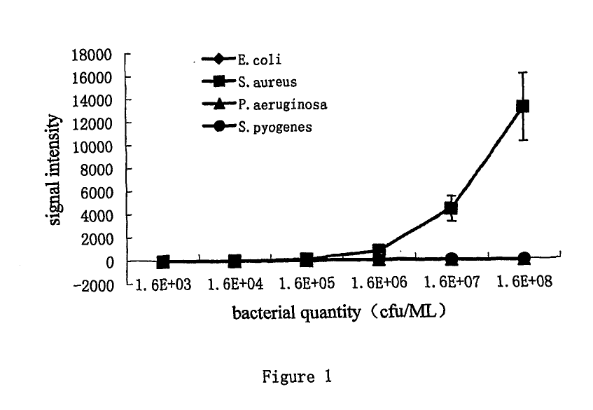

The result of hybridization was shown in FIG. 1.

F represents fluorescent signal on each capture probe. The threshold was defined as the sum of background and three times of the standard deviation, i.e., threshold=Background+3SD. F value greater than threshold indicates a positive signal.

When the concentration of 1 mL S. aureus overnight cultures was 1.6×10⁸, 1.6×10⁷, 1.6×10⁶ and 1.6×10⁵, the fluorescent signals of species-specific capture probes for E. coli, P. aeruginosa and S. pyogenes were all smaller than threshold; but the F value of S. aureus species-specific probe was greater than threshold, and the differences between the F value and the threshold were 3114.0444, 4323.384714, 738.1839105 and 33.73486285, respectively. The detection limitation of S. aureus was 1.6×10⁵ on microarrays using the prevent method.

The bacterial detection needs more than 5-7 days using conventional methods in hospital. However, in the present example, it only took 1.5 hr to get accurate results by hybridizing directly the cell lysate with probes on microarrays. In addition, the detection sensitivity of the present example, is as high as 10⁵ cfu/mL, which is helpful to the rapid diagnosis and proper treatment of patients.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for detecting a target nucleic acid molecule, said method comprises:
a) preparing a cell lysate comprising lysing a cell in a biological sample in a lysis buffer to release the target nucleic acid molecule from the cell;
b) incubating the cell lysate from step a), without nucleic acid purification, with a nucleic acid probe immobilized on a solid substrate under conditions that allow hybridization between the target nucleic acid molecule and the probe, wherein the nucleic acid probe comprises a sequence complementary to the target nucleic acid molecule;
c) assessing hybridization between the target nucleic acid molecule and the probe to determine the presence, absence and/or amount of the target nucleic acid molecule.

2. The method of claim 1, wherein the cell is lysed in the lysis buffer by a physical method.

3. The method of claim 2, wherein the physical method is selected from the group consisting of grinding, ultrasonic lysing, lysing with high temperature, and freezing.

4. The method of claim 1, wherein the cell is lysed in the lysis buffer by a chemical method.

5. The method of claim 4, wherein the chemical method is lysing with a protein denaturant or a detergent.

6. The method of claim 1, wherein the cell is lysed in the lysis buffer by a biological method.

7. The method of claim 6, wherein the biological method is lysing with a proteinase or a lysozyme.

8. The method of claim 1, wherein the cell is lysed by any combination of a physical, a chemical, and a biological method.

9. The method of claim 1, wherein the cell lysate is incubated with the probe immobilized on the substrate in the lysis buffer for hybridization.

10. The method of claim 1, wherein an agent that aids for hybridization is added to the cell lysate before the cell lysate is incubated with the probe.

11. The method of claim 10, wherein the agent is selected from the group consisting of NaCl, citrate sodium, and SDS.

12. The method of claim 1, wherein the biological sample is a sample selected from the group consisting of a non-virus biological organism, a biological tissue, a eukaryotic cell, and a prokaryotic cell.

13. The method of claim 1, wherein the target nucleic acid molecule is selected from the group consisting of a genomic DNA, a plasmid, a mitochondria DNA, a chloroplast DNA, a messenger RNA, a ribosomal RNA, and a small nuclear RNA.

14. The method of claim 1, wherein the solid substrate comprises a material selected from the group consisting of a nylon film, a pyroxylin film, a silicon, a glass, a ceramic, a metal, a plastic, and a combination thereof.

15. The method of claim 1, wherein the solid substrate comprises a plurality of nucleic acid probes, and wherein the plurality of the nucleic acid probes are immobilized on the solid substrate to form an array.

16. The method of claim 15, wherein the plurality of the nucleic acid probes have different nucleotide sequences.

17. The method of claim 16, wherein the number of different probes is from about 2 to about 100,000.

18. The method of claim 15, wherein the area of the array is from about 0.01 mm² to about 100 cm².

19. The method of claim 15, wherein the array is selected from the group consisting of a two-dimensional array, a three-dimensional array, and a four-dimensional array.

20. The method of claim 1, wherein the nucleic acid probe immobilized on the solid substrate comprises a single-stranded oligonucleotide or a double-stranded PCR product.

21. The method of claim 1, wherein the cell lysate comprises an agent selected from the group consisting of a detergent, a protein denaturant, a buffer, a nuclease inhibitor, a salt, and a combination thereof.

22. The method of claim 1, wherein the hybridization between the target nucleic acid molecule and the nucleic acid probe is assessed by determining binding of a reporter to the target nucleic acid molecule, wherein the reporter comprises a detectable marker selected from the group consisting of a fluorescein, an isotope, a biotin, a digoxin, a gold colloid, a magnetic bead, a electrochemical label, and a chemiluminescent label.


SEQUENCE LISTING (8 sequences)

SEQ ID NO: 1 - DNA, 29 bp, Artificial Sequence
  Synthetic construct
ttttttttttttgtattaactttactccc

SEQ ID NO: 2 - DNA, 25 bp, Artificial Sequence
  Synthetic construct
ttcctccccgctgaaagtactttac

SEQ ID NO: 3 - DNA, 29 bp, Artificial Sequence
  Synthetic construct
ttttttttttttagcaagcttctcgtccg

SEQ ID NO: 4 - DNA, 25 bp, Artificial Sequence
  Synthetic construct
ttcgctcgacttgcatgtattaggc

SEQ ID NO: 5 - DNA, 28 bp, Artificial Sequence
  Synthetic construct
ttttttttttttgcgcccgtttccggac

SEQ ID NO: 6 - DNA, 27 bp, Artificial Sequence
  Synthetic construct
gttatcccccactaccaggcagattcc

SEQ ID NO: 7 - DNA, 29 bp, Artificial Sequence
  Synthetic construct
ttttttttttttattactaacatgcgtta

SEQ ID NO: 8 - DNA, 24 bp, Artificial Sequence
  Synthetic construct
gtctctcttatgcggtattagcta